(19) United States
(12) Patent Application Publication
(10) Pub. No.: US 2005/0095278 A1
(43) Pub. Date: May 5, 2005

(54) Use of nitroglycerin to relieve nocturnal muscle cramps

(76) Inventor: Viorel Nicolaescu, New York, NY (US)

Correspondence Address: KIRKPATRICK & LOCKHART NICHOLSON GRAHAM LLP, 535 SMITHFIELD STREET, PITTSBURGH, PA, 15222, US

(21) Appl. No.: 10/341,652

(22) Filed: Jan. 14, 2003

(51) Int. Cl. A61K 9/70; A61K 31/21

(52) U.S. Cl. 424/449; 514/509

(57) ABSTRACT

A method of treating nocturnal muscle cramps is provided. The method includes the step of administering to a patient with nocturnal muscle cramps an amount of nitroglycerin effective to relieve one or more symptoms of the nocturnal muscle cramps. The nitroglycerin typically is delivered topically, substantially superficial to the affected muscle, and most typically by a transdermal patch.

### CROSS REFERENCE TO RELATED APPLICATIONS

Not Applicable.

### BACKGROUND

A method of treating nocturnal muscle cramps is provided. The syndrome of nocturnal muscle cramps affects people of all ages but is particularly prevalent in the elderly, in pregnant women and in athletes. The muscles most often affected are in the calf and the foot. nocturnal muscle cramps are sudden, involuntary painful contractions of the affected muscles which may last from a few seconds up to about 10 minutes.

The etiology of nocturnal muscle cramps is obscure and it is a diagnosis of exclusion which is reached after other known causes of leg pain, such as muscular trauma and neurologic disease, are ruled out. Proposed precipitating factors include heat, sodium depletion, low serum calcium, magnesium or potassium, hemodialysis treatment, age and strenuous exercise.

Treatment options of demonstrated value for nocturnal muscle cramps include physical therapy, including stretching and massage, quinine preparations, vitamin E and verapamil. These are only partially effective and, in the case of quinine, may be associated with significant complications such as hypersensitivity reactions and thrombocytopenia.

### SUMMARY

A method is provided for treating nocturnal muscle cramps. The method includes the step of administering to a patient with nocturnal muscle cramps an amount of nitroglycerin effective to relieve one or more symptoms of the nocturnal muscle cramps, including pain and/or cramping. The nitroglycerine typically is delivered topically, for example and without limitation, by a transdermal device such as a transdermal patch, or by an ointment or lotion, superficial to the affected muscle.

### DETAILED DESCRIPTION

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of these ranges is intended as a continuous range including every value between the minimum and maximum values.

It has been observed that topically applied nitroglycerin, applied by a transdermal patch to the lower extremity, is effective in relieving pain and cramping associated with nocturnal muscle cramps. The appeal of this treatment is that it is site-specific and therefore may avoid the complications of systemically administered medication, and that it acts in a clinically-meaningful time period.

The method includes the step of administering to a patient with nocturnal muscle cramps an amount of nitroglycerin effective to relieve one or more symptoms of the nocturnal muscle cramps. A patient “with nocturnal muscle cramps” is a patient who often experiences nocturnal muscle cramps and, includes, but is not limited to a patient experiencing an acute muscle cramp. Symptoms primarily associated with nocturnal muscle cramps include pain and/or cramping. The nitroglycerine typically is delivered topically, for example and without limitation as an ointment, or through use of a transdermal patch and superficial to (above) the affected muscle. The nitroglycerine preferably is delivered topically through a transdermal device as are well known in the art and are typically passive transdermal devices (patches) as opposed to active devices such as iontophoresis, electroendosmosis or electrophoresis transdermal devices. Passive transdermal devices for the delivery of nitroglycerin are described for example in U.S. Pat. Nos. 5,302,395, 5,262,165 and 5,126,144. Nitroglycerin patches are commercially available under the trade names, for example: MINITRAN (3M) and NITRO-DUR (Schering-Plough).

The therapeutic dosage of nitroglycerin varies from patient-to-patient. Early studies (see below) have shown that delivery of nitroglycerin transdermally at a rate of about 0.1 milligrams per hour (mg/hr) is sufficient for relief of pain and cramping associated with muscle cramps. However, different doses are expected to be effective in different cases, with transdermal dosage ranges from about 0.01 mg/hr to about 1.0 mg/hr, preferably from about 0.1 mg/hr to about 0.8 mg/hr transdermally expected to be effective in many cases. In any case, the optimal and appropriate effective nitroglycerin dosage and dosage form likely will differ from patient-to-patient, and can be readily tailored by altering the patient's dosage and determining the degree of relief from nocturnal muscle cramps. For example, when a transdermal patch is used to deliver the nitroglycerin, the surface area of the patch can be adjusted to alter the drug flux across the skin simply by dividing the patch in half, thirds, quarters, etc., as is illustrated in the examples below. Alternately, the drug flux can be adjusted by providing a transdermal patch with a different flux rate. As used herein, “flux” and “flux rate” refers to the rate of drug delivery through the skin or other membrane by a transdermal device. The flux rates described herein are determined by and are made in reference to industry-standard methods for transdermal product label claims.

Dosage forms other than transdermal patches may be used to the same effect. Commercially available nitroglycerin ointments can be of substantial use. In one example, an ointment is available having 2% nitroglycerin and lactose in a lanolin and white petrolatum base. Other topical dosage forms, such as creams, salves, lotions, gels, sprays and the like are well known in the pharmaceutical arts and would be applicable for use in controlling nocturnal muscle cramps. One example of a topical cream containing nitroglycerin is provided in U.S. Pat. No. 5,698,589. Other nitroglycerin dosage forms are commercially available, such as extended release oral tablets, sublingual tablets, a lingual aerosol spray and even an intravenous dosage form. Each of these formulations may be more or less practicable for any given patient. In most cases, a nitroglycerin patch or ointment applied substantially superficial to the site of nocturnal muscle cramping would be preferable to systemic delivery via oral, lingual, sublingual and certainly intravenous delivery. Nitroglycerin formulations, and variations thereof are well known to those of skill in the art, as is their preparation.

### EXAMPLES

The following Examples show the successful use of topically applied nitroglycerin in treating ten patients with nocturnal leg muscle cramps. The degree of relief from the nocturnal leg cramps is described as follows, with “no relief” meaning the patient noticed no difference in the symptoms (pain and cramping) attributable to the nocturnal leg cramps, “excellent relief” meaning complete resolution of symptoms, “good relief” meaning meaningful improvement in symptoms, but with the need to use the transdermal device more often than those experiencing excellent relief (it was found that certain patients experiencing excellent relief and taking nitroglycerine on an as needed (prn) basis often could skip one or more days between treatment) and “moderate relief” meaning some improvement in symptoms, but with the need to use the transdermal device more often than those experiencing excellent relief.

Patient 1

Patient 1 was a 56 year-old male with a history of obstructive pulmonary disease, hypertension and peripheral vascular disease, along with nocturnal leg cramps. At the time of treatment for leg cramps, this patient was also taking telmisartan and buspirone for other medical conditions. One half of a 0.2 mg/hour NITRO-DUR (Schering-Plough) patch (0.1 mg/hr nitroglycerin) was applied to the patient's skin above the affected leg muscle once a day before bedtime for 10 days with good relief of the nocturnal cramping symptoms.

Patient 2

Patient 2 was an 80 year-old female with a history of gastro-esophageal reflux disease, hypertension and osteoarthritic knee, along with nocturnal leg cramps. At the time of treatment for leg cramps, this patient was also taking metoprolol succinate, triamterene/hydrochlorothiazide, omeprazole and lorazepam for other medical conditions. One half of a 0.2 mg/hour NITRO-DUR patch (0.1 mg/hr nitroglycerin) was applied to the patient's skin above the affected leg muscle once a day for 14 days with moderate-to-good relief of the nocturnal cramping symptoms.

Patient 3

Patient 3 was an 89 year-old female with a history of hypertension, ischemic heart disease, and non-insulin-dependent diabetes mellitus, along with nocturnal leg cramps. At the time of treatment for leg cramps, this patient was also taking glipizide, enalapril maleate, omeprazole and aspirin for other medical conditions. One 0.2 mg/hour NITRO-DUR patch (0.2 mg/hr nitroglycerin) was applied to the patient's skin above the affected muscle once a day as needed for 14 days with moderate-to-good relief of the nocturnal cramping symptoms.

Patient 4

Patient 4 was a 68 year-old female with a history of bronchial asthma, hypertension, congestive heart failure, peripheral vascular disease and hyperthyroidism, along with nocturnal leg cramps. At the time of treatment for leg cramps, this patient was also taking levothyroxine, isosorbide dinitrate, amlodipine besylate, furosemide and spironolactone for other medical conditions. One half of a 0.2 mg/hour NITRO-DUR patch (0.1 mg/hr nitroglycerin) was applied to the patient's skin above the affected muscle once a day as needed for 30 days with good relief of the nocturnal cramping symptoms.

Patient 5

Patient 5 was a 56 year-old female with a history of non-insulin-dependent diabetes mellitus, hypertension, osteoarthritic knee, depression and restless legs, along with nocturnal leg cramps. At the time of treatment for leg cramps, this patient was also taking metformin, fosinopril, celecoxib, tramadol and fluvastatin for other medical conditions. One half of a 0.2 mg/hour NITRO-DUR patch (0.1 mg/hr nitroglycerin) was applied to the patient's skin above the affected muscle once a day as needed for 30 days with excellent relief of the nocturnal cramping symptoms.

Patient 6

Patient 6 was a 68 year-old female with a history of non-insulin-dependent diabetes mellitus, hypertension, osteoarthritic knee and varicose veins, along with nocturnal leg cramps. At the time of treatment for leg cramps, this patient was also taking atenolol, quinapril and glyburide for other medical conditions. One half of a 0.2 mg/hour NITRO-DUR patch (0.1 mg/hr nitroglycerin) was applied to the patient's skin above the affected muscle once a day as needed for 30 days with excellent relief of the nocturnal cramping.

Patient 7

Patient 7 was an 83 year-old female with a history of hypertension, hypercholesterolemia and ischemic heart disease, along with nocturnal leg cramps. At the time of treatment for leg cramps, this patient was also taking metoprolol succinate, fosinopril and aspirin for other medical conditions. One half of a 0.2 mg/hour NITRO-DUR patch (0.1 mg/hr nitroglycerin) was applied to the patient's skin above the affected muscle once a day as needed for 30 days with excellent relief of the nocturnal cramping symptoms.

Patient 8

Patient 8 was an 89 year-old male with a history of hypertension, atrial fibrillation, non-insulin-dependent diabetes mellitus and congestive heart failure, along with nocturnal leg cramps. At the time of treatment for leg cramps, this patient was also taking metoprolol, amlodipine besylate, warfarin, prazosin and folic acid for other medical conditions. One half of a 0.2 mg/hour NITRO-DUR patch (0.1 mg/hr nitroglycerin) was applied to the patient's skin above the affected muscle once a day for 20 days with excellent relief of the nocturnal cramping symptoms.

Patient 9

Patient 9 was a 76 year-old female with a history of hypertension, atherosclerotic heart disease and OA-Knee, along with nocturnal leg cramps. At the time of treatment for leg cramps, this patient was also taking fosinopril, metoprolol, carvedilol and furosemide for other medical conditions. One half of a 0.2 mg/hour NITRO-DUR patch (0.1 mg/hr nitroglycerin) was applied to the patient's skin above the affected muscle once a day for 30 days with good relief of the nocturnal cramping symptoms.

Patient 10

Patient 10 was a 74 year-old female with a history of non-insulin-dependent diabetes mellitus, hypertension and peripheral vascular disease (post bypass surgery), along with nocturnal leg cramps. At the time of treatment for leg cramps, this patient was also taking amlodipine besylate, irbesartan, clonidine, metoprolol and atorvastatin for other medical conditions. One half of a 0.2 mg/hour NITRO-DUR patch (0.1 mg/hr nitroglycerin) was applied to the patient's skin above the affected muscle once a day as needed for 30 days with good relief of the nocturnal cramping.

The data presented above indicates that use of small amounts of nitroglycerin even once daily results in moderate to significant relief in the frequency and severity of nocturnal muscle cramping. From these data, it is expected that variation of the dose of nitroglycerin within standard dosages of nitroglycerin (for example, 0.1 mg/hr to 0.8 mg/hour for commercially-available transdermal nitroglycerin patches), and even lower or higher doses would be effective in treating nocturnal muscle cramping. Further, the dosage may be tailored for each individual to optimize relief.

The above invention has been described with reference to the preferred embodiment. Obvious modifications and alterations will occur to others upon reading and understanding the preceding detailed description and the claims. It is intended that the invention be construed as including all such modifications and alterations.

1. A method of treating nocturnal muscle cramps comprising the step of administering to a patient with nocturnal muscle cramps an amount of nitroglycerin effective to relieve one or more symptoms of the nocturnal muscle cramps.

2. The method of claim 1, comprising topically applying the nitroglycerin to skin substantially superficial to a muscle affected by the nocturnal muscle cramps.

3. The method of claim 2, wherein the nitroglycerin is delivered from a transdermal device.

4. The method of claim 3, wherein the transdermal device is a transdermal patch.

5. The method of claim 4, wherein the flux of nitroglycerin from the patch ranges from about 0.01 mg/hr to about 1.0 mg/hr.

6. The method of claim 4, wherein the flux of nitroglycerin from the patch ranges from about 0.1 mg/hr to about 0.8 mg/hr.

7. The method of claim 4, wherein the flux of nitroglycerin from the patch is about 0.1 mg/hr.

8. The method of claim 2, wherein the nitroglycerin is applied to the skin in one of an ointment or a cream.

9. The method of claim 1, wherein the muscle cramps are leg cramps.

10. The method of claim 1, wherein the symptom is one of pain and cramping.

11. The method of claim 1, wherein the nitroglycerin is delivered orally or sublingually.
